(19) United States
(12) Patent Application Publication
(10) Pub. No.: US 2023/0381090 A1
(43) Pub. Date: Nov. 30, 2023

(54) HAIR, BEARD, MUSTACHE, EYELASH AND EYEBROW CARE FORMULATION

(71) Applicant: Cansu DOGAN, Beykoz/Istanbul (TR)

(72) Inventor: Cansu DOGAN, Beykoz/Istanbul (TR)

(21) Appl. No.: 18/030,770

(22) PCT Filed: Nov. 16, 2020
(86) PCT No.: PCT/TR2020/051112

§ 371 Date: Apr. 7, 2023

(30) Foreign Application Priority Data
Oct. 12, 2020 (TR) 2020/16191

(51) Int. Cl. A61K 8/9789; A61K 8/67; A61K 8/9767; A61Q 5/00

(52) U.S. Cl. CPC A61K 8/9789; A61K 8/678; A61K 8/9767; A61Q 5/00; A61K 2800/591

(57) ABSTRACT

The invention is related to the herbal mix used by the cosmetics sector for hair, beard, eyebrow, eyelash and mustache care. The invention can be applied externally on its own or used with shampoo or similar cleansers and cleaning products.

### TECHNICAL FIELD

The invention is related to the herbal mix used by the cosmetics sector for hair, beard, eyebrow, eyelash and mustache care.

### BACKGROUND OF INVENTION

There are hairs, beards, eyebrows, eyelashes and mustaches on human heads (hairs on the head) with different concentrations depending on gender. Individuals can design these hairs in terms of length, color or other parameters depending on gender differences and for beauty and appearance purposes.

Herbal or chemical shampoos and/or mixes are used in order to have stronger, shinier, fuller and more vibrant hairs on the head. Today, there are many different products available for this purpose. These products are used separately or as mixtures during washing or with external methods.

Hair loss can occur and darker hair can turn grey on certain stages of people's lives or due to various illnesses and treatments. Local or complete hair loss is a natural phenomenon. Hair loss alters the appearance of people and it may cause emotional issues. Today, many people use chemical and herbal products in order to stop hair loss or regrow hair. Chemical products can be harmful for the body and they may have various side effects. Herbal solutions, on the other hand, usually fail to solve the problem or cause problems due to difficult application processes. In addition, hair transplantation by transferring new hair follicles to balding areas is another solution for growing hair after complete hair loss. As hair transplantation is a long and painful process, it is not preferred by everyone.

People with grey hair feel better after dyeing their hair with their preferred colors. Hair is not only dyed for the purpose of coloring grey hair but also for wearing different tints of hair colors. This brings along problems such as weakening and damaging of hair or causing brittle and burnt hair due to various chemicals inside the dye.

Eyelashes are individual hairs on the eyes that prevent particles from entering the eye with the help of eyelid movements. It is considered natural to lose eyelashes or have split eyelashes as the eyelash hairs have a sensitive structure. Especially women use various cosmetic products and nourishing serums to have longer, fuller and stronger eyelashes in order to achieve the desired look. Said serums and cosmetic products mostly consist of chemical ingredients.

In addition to hair, similar problems also apply to facial hairs such as mustache and beard, as well as eyelashes and eyebrows, depending on gender.

Background of invention includes the Chinese patent application No. CN1099666181. This invention contains Wheat Germ Oil, Sweet Almond Oil, Rosemary Oil, Pelargonium Roseum Flower Oil, Lavender Oil, Rose Oil, Bergamot Orange Oil, Tocopheryl Acetate, Jasmine Flower Oil, herbal hair care essential oil prepared by Chinese arborvitae branch and Snakeroot. The invention describes mixing and preparation methods of various endemic herbal oils.

European patent application No. EP0518192 describes hair protection mixture containing lemon juice, olive oil juice, water, parsley juice, celery leaf and root juice, dandelion leaf juice, peeled kiwi juice, yogurt, seedless green olive juice and sea salt.

Both patents described above summarizes the background of the invention but they are only included in patent applications for hair applications. In addition, the products included in said patent applications as well as the background of inventions fail to solve all problems this invention aims to resolve.

### TECHNICAL PROBLEMS SOLVED BY THE INVENTION AND ANALYSES THEREOF

The invention is a completely herbal mixture solving all problems that other products described under the background of invention section fail to resolve.

Although chemical mixtures or shampoos used for caring and repairing hairs on the head such as hair, beards, mustaches, eyelashes and eyebrows give a more vibrant look to the hair in the short term, they may cause weak hair roots, split hairs and hair loss in the long term. In addition, use of different types and doses of products for different regional applications cause difficulty in application and high costs. The invention only consists of herbal oils. The literature shows that the oils individually do not cause any damages to human health or the environment. The invention can be applied on all hairs on the head and makes the hairs more vibrant, stronger and shinier, prevents possible hair loss or ensures hair regrowth.

Certain stages of human lives as well as some illnesses and treatments thereof may cause hair loss on the head. Such hair loss usually seen on the hair. People prefer chemical and herbal mixes for hair regrowth or refer to hair transplantation which is the surgical transfer of hair follicles to balding areas on the head. Said chemical products used for cosmetic purposes, just like all other chemical substances used for human body, fail to resolve these issues or cause damages despite solving these issues. Hair transplantation, on the other hand, is not usually preferred due to high costs of operation, pain experienced during application and unnatural look after application. In addition, it is common to lose transplanted hairs or fail achieving desired natural look. Although these are commonly accepted issues, they are not usually preferred by people. Meanwhile, herbal mixes generally fail to achieve hair regrowth. This invention grows hair that is partially or completely lost due to various reasons without causing any side effects. The invention does not require an external application. The invention is applied by directly using the mixture on the head or adding certain amounts of the mixture to the shampoo. Thus, natural hair regrowth is stimulated and natural look is achieved.

Although dyeing and frequently dressing hair with high heat, using chemicals to clean, design or care hair do not cause major hair loss, they cause heat damage on the hair, weaken the hair and cause the hair to lose its natural look. People usually prefer external care and repairing products in order to prevent these damages. Such products offer a short-term solution for these problems. However, they cause recurrence of the same problems or hair loss in the long term. The invention can be used separately or added to individuals' shampoo products depending on the frequency of washing the hair. Therefore, the damages caused throughout the day can be prevented and the hair care can be made without additional effort or product use.

### Technical Specifications of the Invention and Descriptions Thereof

The invention is related to the herbal mix used by the cosmetics sector for hair, beard, eyebrow, eyelash and mustache care.

Said hair care and repair formulation are given in the detailed description of the invention without any limiting effects and for the purposes of clarification of the subject matter.

The invention is sampled as a completely herbal mixture and there are not any limitations for being used separately, added to herbal or chemical shampoos or produced as a shampooing product.

The invention, which is a hair, beard, eyebrow, eyelash and mustache care formulation, contains 25-35% Prunus Amygdalus Dulcis Oil (Sweet Almond Oil) in volume, 10-25% Pinus Pinaster-Turpentine (Pine Turpentine) in volume, 25-40% Nigella Sativa Seed Oil (Nigella Oil) in volume, 10-25% Sesamum Indicum Seed Oil (Sesame Seed Oil) in volume, Tocopherol (Natural Vitamin E) in volume.

Functions of the raw materials used in formulation of this invention are explained below:

Sweet Almond Oil: The oil speeds up cell rejuvenation thereby ensuring faster hair growth and growing thicker hair with Vitamins A and K it contains. It regenerates irritated parts of the skin and moisturizes the skin and the hair.

Sesame Seed Oil: The oil stimulates cell rejuvenation thereby strengthening hair follicles, thickening strands of hair and increasing hair growth rate with Vitamins A, B and E it contains. The fatty acids prevent dandruffs, luster hair and repair damaged ends. It moisturizes hair and the skin.

Pine Turpentine: Strengthens hair follicles thereby increasing hair growth rate and reducing hair loss. It balances sebum generation on the scalp.

Natural Vitamin E: Moisturizes hair, makes the hair flexible, repairs damages due to external factors and gives resistance to hair. Balances pH levels of hair and regulates sebum generation. Stimulates hair follicles and increases hair growth rate.

Nigella Oil: Contains thymoquinone, nigellon, myristic acid, palmitic acid, stearic acid, proteins, Vitamins B1, B2 and B3, and minerals such as calcium, phosphate, iron, is copper. Prevents hair from turning grey early, ensures growth of new hairs, healthy hair growth and voluminous look and prevents hair loss and dandruffs.

1. The invention is related to the herbal mix used by the cosmetics sector for hair, beard, eyebrow, eyelash and mustache care and contains the following;
25-35% Prunus Amygdalus Dulcis Oil (Sweet Almond Oil) in volume,
10-25% Pinus Pinaster-Turpentine (Pine Turpentine) in volume,
25-40% Nigella Sativa Seed Oil (Nigella Oil) in volume,
10-25% Sesamum Indicum Seed Oil (Sesame Seed Oil) in volume,
0.10-0.4% Tocopherol (Natural Vitamin E) in volume.
